(19) United States
(12) Patent Application Publication
(10) Pub. No.: US 2007/0054293 A1
(43) Pub. Date: Mar. 8, 2007

(54) MICROFLUIDIC CHAOTIC MIXING SYSTEMS AND METHODS

(75) Inventors: Jian Liu, Menlo Park, CA (US); Brian A. Williams, Pasadena, CA (US); Barbara J. Wold, San Marino, CA (US); Stephen Quake, Stanford, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: California Institute of Technology, Pasadena, CA (US)

(21) Appl. No.: 11/468,642

(22) Filed: Aug. 30, 2006

Related U.S. Application Data
(60) Provisional application No. 60/712,676, filed on Aug. 30, 2005.

(51) Int. Cl. C12Q 1/68; C12M 1/34

(52) U.S. Cl. 435/6; 435/287.2

(57) ABSTRACT

Microfluidic nucleic acid hybridization systems are described that include a first reaction chamber to hold an analyte solution comprising nucleic acids, and a first mixing channel in fluid communication with the chamber. The mixing channel includes a textured surface to mix the analyte solution. The systems may also include pump coupled to the mixing channel to circulate the analyte solution through the reaction chamber and the mixing channel, and an input port in fluid communication with the mixing channel and the reaction chamber to supply the analyte solution to the microfluidic system. The input port can be closed to create a closed circulation path for the analyte solution through the reaction chamber and the mixing channel.

### CROSS-REFERENCES TO RELATED APPLICATIONS

This application claims priority to U.S. Provisional App. No. 60/712,676, filed Aug. 30, 2005, and titled “Enhanced Signals and Fast Nucleic Acids Hybridization Using Microfluidic Chaotic Mixing”, the entire contents of which are herein incorporated by reference for all purposes.

### STATEMENT AS TO RIGHTS TO INVENTIONS MADE UNDER FEDERALLY SPONSORED RESEARCH OR DEVELOPMENT

Work described herein has been supported, in part, by the NIH Director's Pioneer Award, 7DP 1 0D000251-02. The work has also been supported, in part, by National Institutes of Health grant NIH [1 R01 HG002644-01A1]. The United States Government may therefore have certain rights in the invention.

### BACKGROUND OF THE INVENTION

Nucleic acids hybridization techniques have been widely used in both fundamental and clinical research to identify genes and mutants, to map their correlations and analyze their expression. DNA microarrays immobilize thousands of oligonucleotides or cDNA clones or PCR products on the solid substrate, thus providing a powerful tool for large-scale detection of target genes. However, hybridization in conventional microarray experiments is performed in a diffusion-limited manner, which is quite inefficient: The process may take 8 to 24 hours; even so the characteristic length (1-3 mm) that a target DNA molecule can cover is still one order of magnitude less than the typical size of most microarrays (>10 mm).

In microarray experiments, the height of hybridization chambers is typically in the order of dozens of microns. The motion of the fluid within such dimension is dominated by the laminar flow. Essentially, the challenge is to mix the sample solution well and transport the DNA molecules to the proximity of the probes effectively in order to increase the valid molar hybridization events. Several reports introduced the methods of ultrasonically-induced transportation or acoustic microstreaming for mixing. But the predefined geometry of their oscillating sources produced specific flow pattern, therefore the targets in the solution can be constrained in particular regions instead of the whole hybridization area. Other methods also appeared to result in non-well-mixed regions, including alternative convection induced through several ports, “drain and fill” or air driven bladders, and magnetic stirring bars. Some researchers developed electrokinetic methods to accelerate the transportation of DNA molecules, but only a limited number (less than 100) of spots per chip can be detected so far.

Thus, there remains a need for systems for and methods of thoroughly mixing analyte solutions in shorter amounts of time. There is also a need to make these novel mixing systems and methods compatible with nucleic acid hybridization techniques. These and other issues are addressed by embodiments of the present invention.

### BRIEF SUMMARY OF THE INVENTION

Embodiments of the invention include microfluidic nucleic acid hybridization systems that may include a first reaction chamber to hold an analyte solution comprising nucleic acids and a first mixing channel in fluid communication with the chamber. The channel includes a textured surface to mix the analyte solution. The systems may also include a pump coupled to the mixing channel to circulate the analyte solution through the reaction chamber and the mixing channel, and an input port in fluid communication with the mixing channel and the reaction chamber to supply the analyte solution to the microfluidic system. The input port can be closed to create a closed circulation path for the analyte solution through the reaction chamber and the mixing channel.

Embodiments of the invention also include methods of chaotically mixing and hybridizing a nucleic acid solution in a microfluidic system. The methods may include providing the nucleic acid solution to a first reaction chamber, where the reaction chamber contains nucleic acid hybridization sites. The methods may also circulating the nucleic acid solution a plurality of times through a mixing channel coupled to the reaction chamber. The solution is mixed as it flows across a textured surface of the mixing channel to mix the solution. The methods may further include hybridizing nucleic acids in the solution to one of the hybridization sites.

Embodiments of the invention still further include microfluidic nucleic acid hybridization systems that include a glass substrate having an array of nucleic acids on a top surface of the substrate. A first elastomeric layer may be attached to the top surface of the glass substrate, where the elastomeric layer has a first and second reaction chamber formed therein, and where the top surface of the substrate forms an inside surface of each reaction chamber. The systems may also include a mixing channel that is also formed in the first elastomeric layer, where the mixing channel is in fluid communication with the first and second reaction chamber. The mixing channel has a textured surface with a herring-bone pattern to mix a nucleic acid solution flowing through the channel. In addition, a second elastomeric layer may be formed on the first elastomeric layer. The second elastomeric layer may have a series of control channels that activate a microfluidic peristaltic pump in the mixing channel. The systems may also include an input port in fluid communication with the mixing channel and the reaction chamber to supply the nucleic acid solution to the microfluidic system. The input port may be coupled to a closable microvalve that creates a closed circulation path for the nucleic acid solution between the reaction chamber and the mixing channel.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1A is a plan view of a chaotic mixing system according to embodiments of the invention;

FIG. 1B is an optical micrograph of a chaotic mixing device according to embodiments of the invention;

FIG. 1C is another plan view of a chaotic mixing system according to embodiments of the invention;

FIG. 1D is still another plan view of a chaotic mixing system according to embodiments of the invention;

FIGS. 2A-B show graphs of mixing efficiency for a mixing device that has herring bone shaped protrusions in a mixing channel;

FIGS. 2C-D show graphs of mixing efficiency for a mixing device without a turbulence generating pattern in a mixing channel;

FIG. 3 shows a flowchart that includes steps in methods of chaotically mixing and analyzing an analyte solution according to embodiments of the invention;

FIG. 4 shows a flowchart that includes steps in methods of mixing and hybridizing an polynucleotides according to embodiments of the invention;

FIGS. 5A-B show Cy3 labeled cDNA hybridized onto home-spotted microarrays after chaotic mixing and conventional diffusion, respectively;

FIG. 5C shows titration curves of the hybridized cDNA after chaotic mixing and conventional diffusion;

FIG. 5D shows a comparison of the coefficients of variation (CV) of the hybridized cDNA after chaotic mixing and conventional mixing;

FIG. 6 shows fluorescent images of two side by side hybridization experiments using chaotic mixing and conventional diffusion, respectively;

FIG. 7 shows a histogram of background-subtracted fluorescence for Cy3 labeled cDNA hybridization signals measured after microfluidic chaotic mixing and conventional diffusion, respectively;

FIG. 8 shows graphs of the percentage of hybridized CAB molecules compared to the input amount for chaotic mixing and conventional diffusion, respectively.

### DETAILED DESCRIPTION OF THE INVENTION

Microfluidic chaotic mixing systems, devices and methods are described that enhance the mixing efficiency of analyte solutions. By allowing analyte solutions to mix in shorter periods of time, the analytes in solution can contact, bind with and otherwise react with receptor sites (as well as other analytes) in less time. For example, the experimental results described below demonstrate that microfluidic chaotic mixing can enhance hybridization signals of cDNA molecules 3 to 8 fold by introducing lateral mixing, facilitating the delivery of the molecules, and increasing the molar hybridization events. This has improved the detection limit of DNA microarray experiments by nearly one order of magnitude. The time-consuming step of the conventional method has been reduced to 2 hours using embodiments of systems and methods according to the present invention.

The chaotic mixing systems may be disposable, and compatible with home-spotted or commercial high density microarray slides. Devices with larger chambers (e.g., 2 mm×7.5 mm×36 mm) may hybridize more than 135,000 spots in a single experiment when, for example, fountain pen technology is used to spot ultrahigh density microarrays (25,000 spots/cm²).

In addition, the chaotic mixing systems and devices may be used in conjunction with other equipment, such as elastomeric devices for cell lysis and extraction of mRNA in a parallel fashion (see, for example, Hong et al, U.S. patent application Ser. No. 10/678,946, filed Oct. 2, 2003, and titled “MICROFLUIDIC NUCLEIC ACID ANALYSIS”, the entire contents of which is herein incorporated by reference for all purposes). Microfluidic large scale integration and automation of the long procedure of microarray experiments including sample preparation will facilitate gene expression studies and may even change the state of the art in this field.

As described below, in addition to their utility in analysis of nucleic acids (e.g., cDNA and oligonucleotide arrays) systems of the invention may be adapted for use with any ligand-anti-ligand system (where the term “ligand” corresponds to the analyte). Exemplary ligand (analyte)-antiligand pairs include complementary or partially complementary nucleic acids in which one strand is a ligand (analyte) and the other an antiligand; proteins or peptides and protein-binding moieties (e.g., antibodies, antibody fragments, and protein-binding receptors), lectins and sugars, and the like.

Examples of nucleic acid solutions may include solutions having nucleic acid analytes such as RNA (e.g., mRNA), DNA (e.g., cDNA), PNA, hybrid or chimeric nucleic acids. In certain embodiments the nucleic acid analytes may have base pair lengths ranging from about 10 to about 5000 base pairs, about 10 to about 1000 base pairs, or about 20 to about 500 base pairs.

Exemplary Chaotic Mixing Systems and Devices

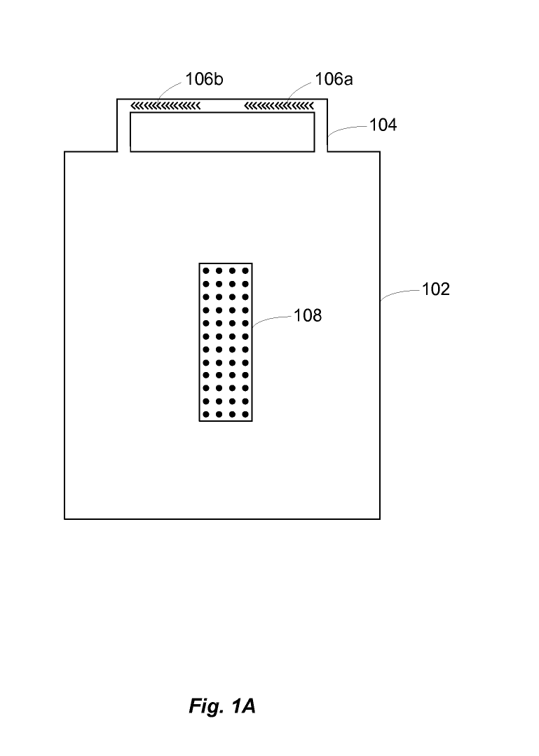

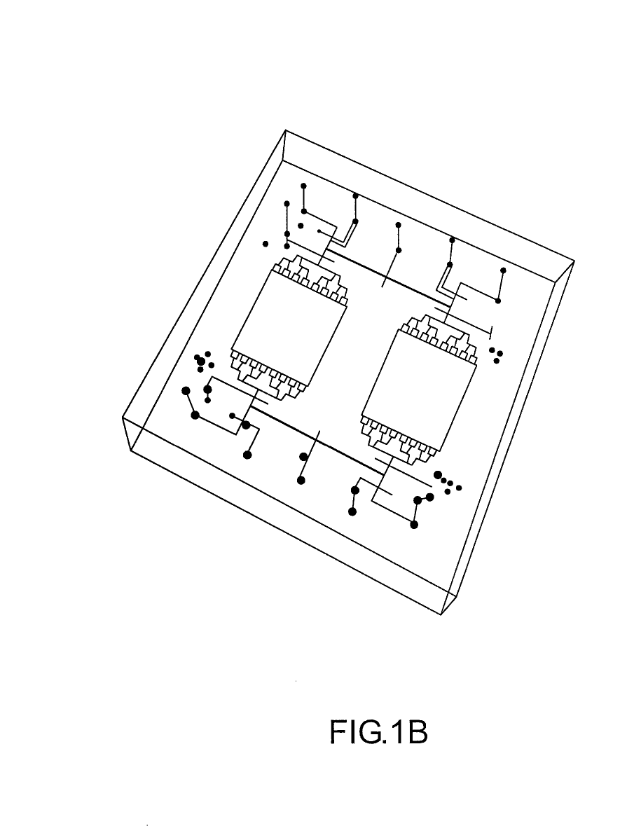

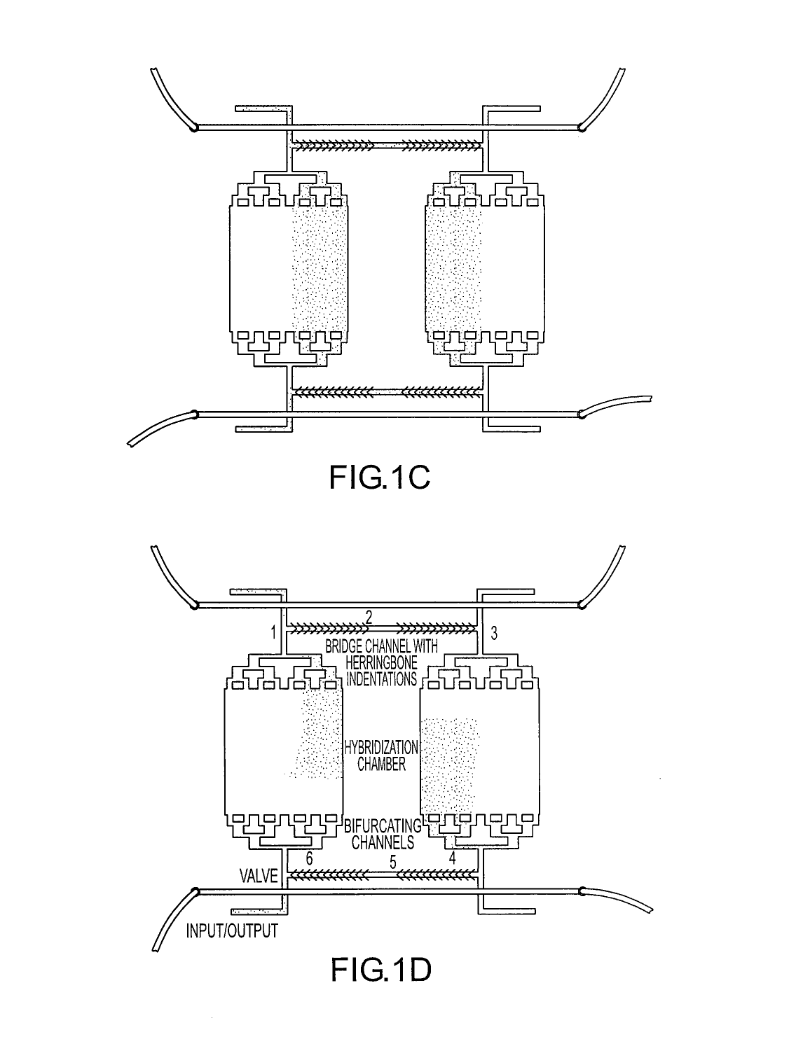

FIG. 1A shows a plan view of a microfluidic chaotic mixing system 100 according to embodiments of the invention. The system 100 includes a chamber 102 that brings and analyte solution (not shown) into contact with analyte biding sites 108 that are contained in the chamber. The analyte binding sites 108 may be formed on a surface of the chamber 102 or may be formed on a separate substrate that coupled to or otherwise placed inside the chamber 102. Systems of the invention may comprise one, two or more than two chambers, some or all of which may contain a plurality of ligands immobilized on a surface of or within the chamber.

Chambers of the system may have a variety of shapes and sizes. For example, the chamber may be circular (e.g., coin shaped), elliptical, triangular, square, rectangular (e.g., box shaped), trapezoidal, or polygonal shaped, among other shapes. The volume (or liquid capacity) can vary widely from the nanoliter to microliter range. In certain embodiments the reactor capacity is less than 10 microliters. In certain embodiments, the volume or liquid capacity of the chamber is greater than 10 microliter, sometimes greater than 50 microliters, and sometimes greater than 100 microliters. In certain embodiments, the volume of the chamber is from 10 to 200 microliters, 20 to 150 microliters, or 50 to 100 microliters.

In the system illustrated in FIG. 1A, the analyte solution can circulate in a closed loop between the chamber 102 and a mixing channel 104 to mix the solution. When there are two or more than two chambers, the chambers may be connected with each other by mixing channels (also called “bridge channels”) that have textured surfaces to promote fluid mixing. For example, the analyte solution may circulate a plurality of times through the mixing channels and from one chamber to another, with each pass through a channel facilitating fluid mixing. The mixing is aided by indentations and/or protrusions (106a-b in FIG. 1) in a textured surface of the mixing channel that mix the passing analyte solution.

A mixing channel may be a microfluidic channel through which a solution can flow. In certain embodiments the channel is formed in an elastomeric material, as described below. The dimensions of mixing channels can vary widely but typically include at least one cross-sectional dimension (e.g., height, width, or diameter) less than 1 mm, preferably less than 0.5 mm, and often less than 0.3 mm. The channels often have at least one cross-sectional dimension in the range of 0.05 to 1000 microns, more preferably 0.2 to 500 microns, and more preferably 10 to 250 microns. The channel may have any suitable cross-sectional shape that allows for fluid transport, for example, a square channel, a circular channel, a rounded channel, a rectangular channel, etc. In an exemplary aspect, the channels are rectangular and have widths of about in the range of 0.05 to 1000 microns, more preferably 0.2 to 500 microns, and more preferably 10 to 250 microns. In an exemplary aspect, the channels have depths of 0.01 to 1000 microns, more preferably 0.05 to 500 microns, more preferably 0.2 to 250 microns, and more preferably 1 to 100 microns. In an exemplary aspect, the channels have width-to-depth ratios of about 0.1:1 to 100:1, more preferably 1:1 to 50:1, more preferably 2:1 to 20:1, and most preferably 3:1 to 15:1, and often about 10:1. The channels in elastomeric devices may have a curved or elliptical face that allows the deflected elastomeric membrane to be fully compliant to the round-profile mixing channel and allowing complete closure of monolithic valves. In one embodiment the mixing channel dimensions are 250-300 microns by 45 microns. In one embodiment the mixing channel dimensions are 400 microns wide and 40 microns high. Although certain preferred embodiments have been described, the mixing channels of the invention are not limited to the dimensions above.

The channels may have at least one textured surface in contact with the fluid. The textured surface may include a plurality of groves and/or protrusions with orientations that form angles relative to a principle fluid flow direction and mix the circulating fluid. For example, the groves and protrusions may being chevron-shaped having a apex that is formed by lines intersecting at an angle. The intersecting lines may be linear or non-linear, as well as symmetrical or asymmetrical. The orientation of the shapes may be at an angle to a principle flow direction of fluid in the mixing channel. The groves and protrusions may also have dimensions that are smaller than the dimensions of the mixing channel in which they are formed. They may be periodically or randomly arranged along the length of the mixing channel.

The textured surfaces may be formed in a variety of geometrical shapes, including rectangular, circular, and parabolic, among others. The shapes may be combined into a periodic or random arrangement in the mixing channel. As noted above, the shapes may include a plurality of chevron-shapes that form a herring-bone pattern. As used herein, the term “herring-bone pattern” has its normal meaning of columns (e.g., two) of short parallel lines with all the lines in one column sloping one way and lines in adjacent column sloping the other way. One example, for illustration and not limitation, is shown in FIG. 1. Additional details about the patterns that may be formed in the textured surfaces to facilitate fluid mixing are described in U.S. Published Patent Application US2004/0262223, titled “LAMINAR MIXING APPARATUS AND METHODS” by Stook et al, the entire contents of which is herein incorporated by reference for all purposes.

Additional examples of systems and methods may include mixing channels having smooth surfaces. These systems primarily rely on closed loop circulation to mix the analyte solution. For example, a single chamber system may circulate the analyte solution through a mixing channel and back to the chamber a plurality of times to mix the solution. A multiple chamber system (e.g., a two chamber system) may circulate the analyte solution through a plurality of bridge channels from one chamber to another. In these examples, the mixing or bridge channels have relatively smooth surfaces that do not create a lot of additional mixing and/or turbulence in the passing fluid.

In some embodiments of the invention, bifurcating channels connect a mixing channel and a chamber (i.e., forming a manifold, see FIG. 1). The manifold is used for distribution of fluid flow into the chamber(s) from the bridge channel(s). A distribution manifold is a configuration of flow channels that serve to divide flow into several parts, with the parts being introduced through different ports into the same reactor. The solution may be introduced equally and simultaneously through the ports. In one embodiment, a manifold for introduction of a solution to the chamber is fashioned generally as shown in FIG. 1D. The manifold allows a solution to simultaneously enter and/or exit the chamber from more locations, which leads to faster mixing and shorter reaction times. Simultaneous introduction of liquid may be accomplished by having equal path lengths in the channel work from the origin of the manifold to each opening to the chamber. Embodiments may also include distribution manifolds having 4-10 ports or more that are equidistant from the first splitting point.

FIG. 1B shows a system made from polydimethylsiloxane (PDMS) that uses integrated peristaltic pumps to circulate an analyte solution between two large chambers, while chaotically mixing the components of the solution in bridge (i.e., mixing) channels. The system can enhance hybridization signals 3 to 8 fold, compared with a conventional static method; and reduce hybridization time to about 2 hours. The system has many benefits over conventional static systems, including higher sensitivity, lower sample volume (5-35 uL), lower cost, compatibility with commercially available microarray slides, and ease of large-scale integration.

As shown in FIGS. 1C-D and described in the examples below, a two-layer PDMS microfluidic device was fabricated and sealed to a spotted microarray slide to perform dynamic hybridization. The fluidic layer of the device contains two symmetric hybridization chambers (6.0 mm×6.5 mm×65 microns). Four input/output through-holes with corresponding micromechanical valves are used for loading sample solutions or disposing waste buffers. Those valves are actuated to form closed chambers during the circulation of the fluid. Additionally, in the control layer two sets of peristaltic pumps are integrated to move the fluid between the hybridization chambers.

The design allows different components in the solution to mix in a chaotic manner when they pass through the bridge channels, then to be delivered through the hybridization chambers, as shown in FIGS. 1A-D. For the devices shown, peristaltic pumps move the analyte solution at a velocity of ˜5.2 nL/sec, taking about 16 minutes to complete one round of circulation between the two chambers. The velocity may be further accelerated by increasing the cross sectional area of the individual pump.

The efficiency of mixing was evaluated by loading the chambers half-to-half with the blank solution and the solution containing fluorescent beads, actuating the pumps, and performing fluorescence measurements. A fluorescent inverted microscope with a home-made CCD camera was set up to take images of the device. Chaotic mixing was confirmed by the observations of the beads' zigzag motion and crossing one another through the bridge channels (images not shown). Ten windows along the equator of one chamber were specified to monitor the fluorescent intensity changes in real-time.

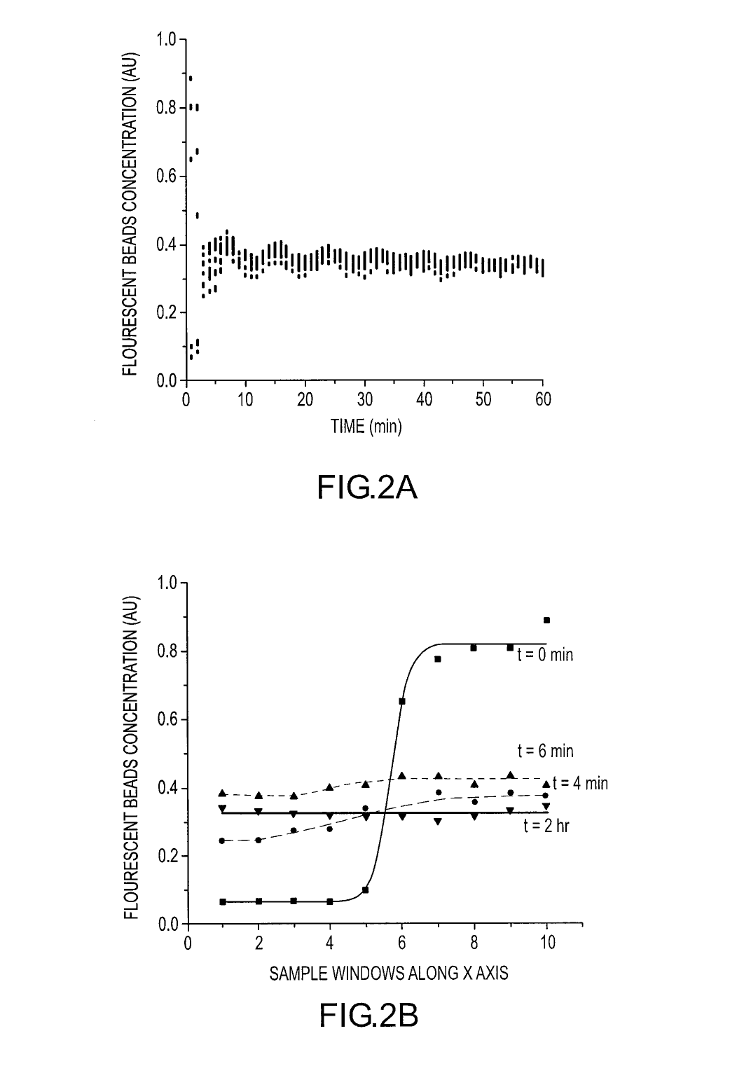

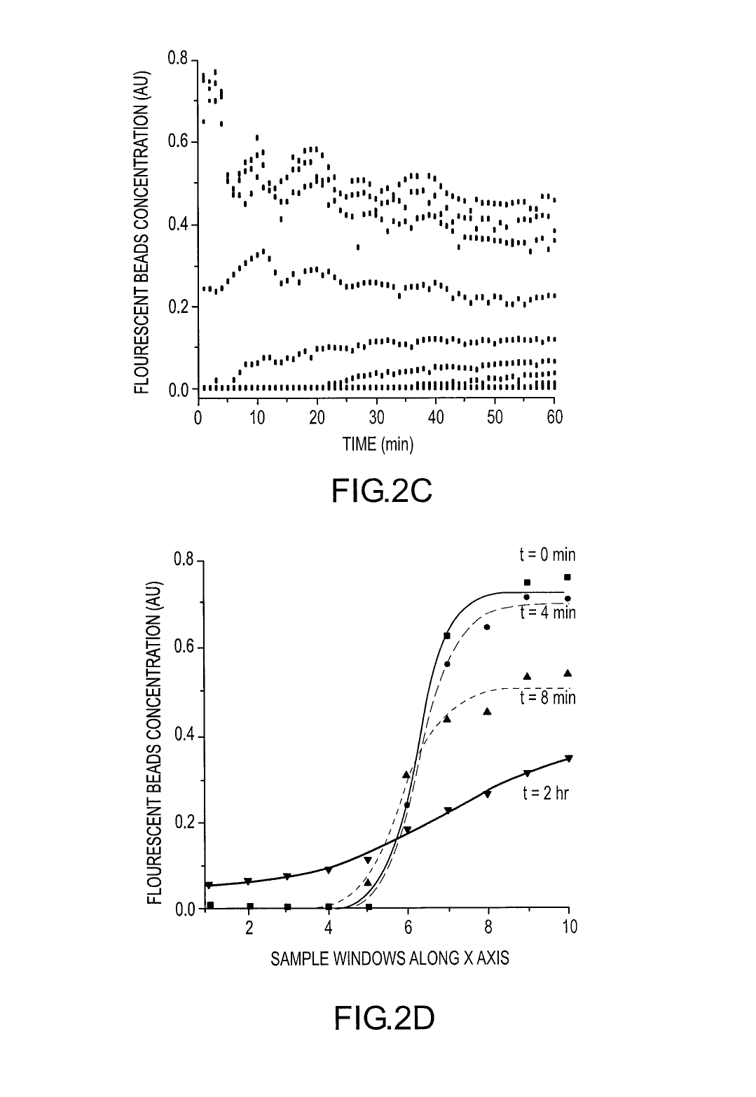

FIGS. 2A-D provide evaluations of the mixing efficiencies of devices with herringbone protrusions in the bridge channels (FIGS. 2A&B) and without such protrusions in the bridge channels (FIGS. 2C&D). The sample-loading pattern was similar to the micrograph FIG. 1A. A solution containing 0.1 micron fluorescent beads and a blank solution were used to replace the red and blue colors, respectively. In FIGS. 2B and 2D, the black squares represent the beginning status (0 min) before circulating the solutions; the red dots (4 min); the green upward triangles (8 min); the blue downward triangles (2 hr). The Stokes-Einstein diffusion coefficient of 0.1 micron beads was estimated to be 4.4×10⁻⁸ cm²s⁻¹, comparable to that of DNA molecules (1000 bp).

As shown in FIG. 2A and FIG. 2B, chaotic mixing reduced the fluorescence gradient along the equator in minutes. The control experiment followed all the same conditions except for using a device without the herring-bone protrusions on the bridge channels. As shown in FIG. 2C and FIG. 2D, the fluorescence difference along the equator was still substantial due to the absence of effective lateral mixing even after 2 hours circulation of the solutions. The results indicated that the herring-bone protrusions enhanced chaotic mixing and the homogenization of the solutions introduced into the chambers. Ripples in fluorescence intensity were observed (FIG. 2A and FIG. 2C) because the extra blank solution initially loaded in the bridge channels took part in the fluid circulation. Interestingly, they became a good index of the periodicity of the circulation (˜8 minutes/ripple).

Elastomeric Devices

The systems and devices of the invention can be made from a variety of materials used in making microfluidic devices. In certain embodiments systems and devices are made from elastomeric polymers. Elastomers in general are polymers existing at a temperature between their glass transition temperature and liquefaction temperature. See Allcock et al., Contemporary Polymer Chemistry, 2nd Ed. and include silicone polymers such as polydimethylsiloxane (PDMS), polytertrafluoroethylene (Teflon) and other materials. Common elastomeric polymers include perfluoropolyethers, polyisoprene, polybutadiene, polychloroprene, polyisobutylene, poly(styrene-butadiene-styrene), polyurethanes, and silicones, for example, or poly(bis(fluoroalkoxy)phosphazene) (PNF, Eypel-F), poly(carborane-siloxanes) (Dexsil), poly(acrylonitrile-butadiene) (nitrile rubber), poly(1-butene), poly(chlorotrifluoroethylene-vinylidene fluoride) copolymers (Kel-F), poly(ethyl vinyl ether), poly(vinylidene fluoride), poly(vinylidene fluoride-hexafluoropropylene) copolymer (Viton), elastomeric compositions of polyvinylchloride (PVC), polysulfone, polycarbonate, polymethylmethacrylate (PMMA), polydimethylsiloxane copolymer, and aliphatic urethane diacrylate.

Methods for fabrication of microfluidic devices and closable microvalves using elastomeric materials are described in, for example, Unger et al., 2000, Science 288:113-16; U.S. Pat. No. 6,960,437 (Nucleic acid amplification utilizing microfluidic devices); U.S. Pat. No. 6,899,137 (Microfabricated elastomeric valve and pump systems); U.S. Pat. No. 6,767,706 (Integrated active flux microfluidic devices and methods); U.S. Pat. No. 6,752,922 (Microfluidic chromatography); U.S. Pat. No. 6,408,878 (Microfabricated elastomeric valve and pump systems); U.S. Pat. No. 6,645,432 (Microfluidic systems including three-dimensionally arrayed channel networks); U.S. Patent Application publication Nos. 20040115838, 20050072946; 20050000900; 20020127736; 20020109114; 20040115838; 20030138829; 20020164816; 20020127736; and 20020109114; PCT patent publications WO 2005084191; WO 2005030822; and WO 200101025; Quake & Scherer, 2000, “From micro to nanofabrication with soft materials” Science 290: 1536-40; Unger et al., 2000, “Monolithic microfabricated valves and pumps by multilayer soft lithography” Science 288:113-116; Thorsen et al., 2002, “Microfluidic large-scale integration” Science 298:580-584; Chou et al., 2000, “Microfabricated Rotary Pump” Biomedical Microdevices 3:323-330; Liu et al., 2003, “Solving the “world-to-chip” interface problem with a microfluidic matrix” Analytical Chemistry 75, 4718-23,” of which the entire contents of all these references are herein incorporated by reference for all purposes.

Microfluidic devices are generally constructed utilizing single and multilayer soft lithography (MSL) techniques and/or sacrificial-layer encapsulation methods. The basic MSL approach involves casting a series of elastomeric layers on a micro-machined mold, removing the layers from the mold and then fusing the layers together. In the sacrificial-layer encapsulation approach, patterns of photoresist are deposited wherever a channel is desired. One exemplary method for fabricating elastomeric devices is briefly described below.

In brief, one method for fabricating elastomeric devices involve fabricating mother molds for top layers (the elastomeric layer with the control channels and reactors, the elastomeric layer with the flow channels) on silicon wafers by photolithography with photoresist (Shipley S J R 5740). Channel heights can be controlled precisely by the spin coating rate. Photoresist channels are formed by exposing the photoresist to UV light followed by development. Heat reflow process and protection treatment is typically achieved as described by Unger et al. supra. A mixed two-part-silicone elastomer (GE RTV 615) is then spun into the bottom mold and poured onto the top mold, respectively. Spin coating can be utilized to control the thickness of bottom polymeric fluid layer. The partially cured top layer is peeled off from its mold after baking in the oven at 80° C. for 25 minutes, aligned and assembled with the bottom layer. A 1.5-hour final bake at 80° C. is used to bind these two layers irreversibly. Once peeled off from the bottom silicon mother mold, this RTV device is typically treated with HCL (0.1N, 30 min at 80° C.). This treatment acts to cleave some of the Si—O—Si bonds, thereby exposing hydroxy groups that make the channels more hydrophilic.

The device can then optionally be hermetically sealed to a support or substrate (which may form a wall of the reaction chamber and flow channels). The substrate can be manufactured of essentially any material (examples of suitable supports include glass, plastics and the like) although the surface should be flat to ensure a good seal. The substrate can comprise a plurality (e.g., array) of ligands adherered to the surface such that then the substrate is sealed to the elastomeric material containing flow channels a chamber is formed having ligands contained therein. For example, cDNAs may be spotted on the surface of a slide, and the flow layer of an elastomeric device may be sealed to the slide so that the cDNAs are within the chamber. Alternatively, the substrate can be an elastomeric material and/or the cDNAs or other ligands can be adhered to a different wall of the chamber Alternatively, an array on a substrate can be placed into the chamber. For example, an oligonucleotide array on a silicon substrate (e.g., such as the GeneChip arrays manufactured by Affymetrix) can be placed in the chamber before the chamber is sealed.

It will be immediately appreciated that an advantage of using elastomeric materials in the practice of the invention is that such materials are readily used to form devices such as those described in Unger et al. and other references cited supra in which monolithic valves and osmotic pumps comprising such valves may be incorporated.

Exemplary Chaotic Mixing Methods

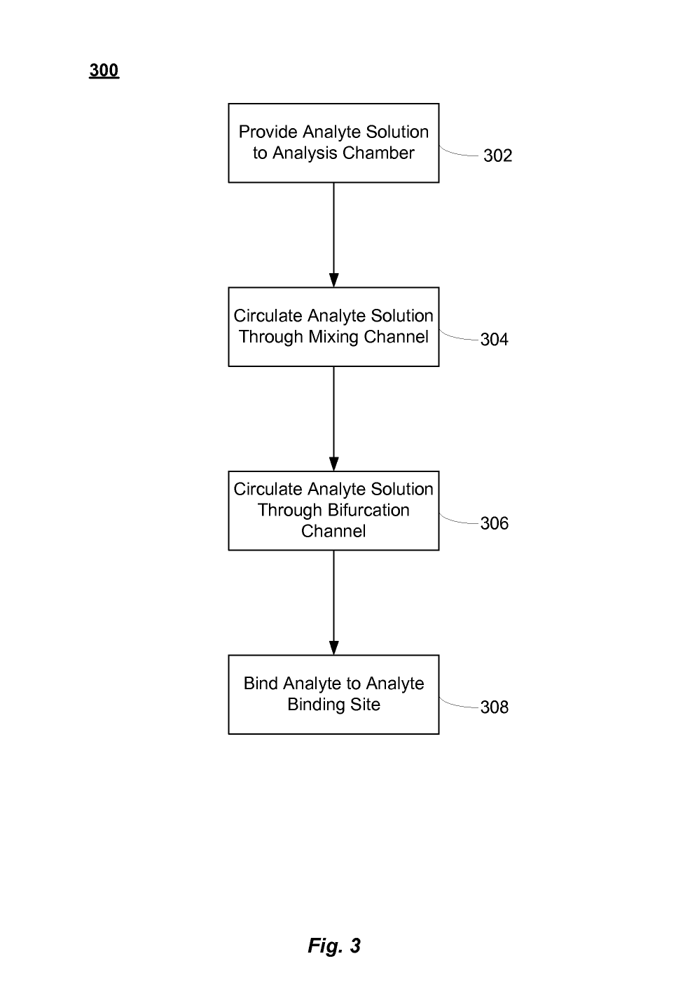

FIG. 3 shows a flowchart that includes steps in an exemplary method 300 of chaotically mixing and analyzing an analyte solution according to embodiments of the invention. The method 300 includes the step of providing an analyte solution to an analysis chamber 302. The analyte solution may include the analyte, as well as organic molecules, inorganic molecules, bioes, salts, buffers, and the like. The analyte solution is designed so the analyte will specifically bind to corresponding ligand in the chamber. The system may also include components to regulate the temperature of the chamber to facilitate hybridization or binding.

The analysis chamber may include one or more chambers fluidly connected to at least one mixing channel. For example, the analysis chamber may include two analyte chambers that are connected by two mixing (i.e., bridge) channels, as shown in FIGS. 1A-C. At least one of the analyte chambers may include an analyte binding area on a surface exposed to the homogenizing analyte solution. The analyte binding area may include a plurality of analyte binding sites, and the binding sites may be designed to bind (e.g., hybridize) with a specific target analyte species. For example, a binding site may include a strand of cDNA having a nucleotide sequence tailored to hybridize with a specific polynucleotide strand in the analyte solution.

The analyte solution provided to the analyte chamber(s) is circulated through at least one mixing channel fluidly coupled to the chamber(s) 304. The mixing channel is configured to cause chaotic mixing of the analyte solution as it circulates through the channel. As noted above, this chaotic mixing may be caused by forming turbulence generating protrusions (i.e., patterns raised above the surface) in one or more surfaces of the channel that makes contact with the analyte solution. In an exemplary embodiment, the turbulence generating protrusions include a plurality of herring-bone shaped objects that are aligned in series along a flow surface of the mixing channel.

A pumping mechanism may cause the analyte solution to flow in a turbulent manner over and around the turbulence generating protrusions causing the solution to become turbulent and undergo chaotic mixing. The active pumping and mixing of the solution increases it level of homogeneity more rapidly than for a solution mixing statically at room temperature. The pumping mechanism used to circulate the analyte solution through the analyte chamber and mixing channel may be a peristaltic pump formed adjacent to the mixing channel. The peristaltic pump may include a series of three or more valves that are selectively opened and closed to create a peristaltic pumping action in the mixing channel. Additional details of a microfluidic peristaltic pump that may be used with the devices and systems of the present invention is described in Unger et al, U.S. Pat. No. 6,408,878, filed Feb. 28, 2001, and titled “MICROFABRICATED ELASTOMERIC VALVE AND PUMP SYSTEMS”, the entire contents of which is herein incorporated by reference for all purposes. Alternatively pumps can be electronic, electrostatic, magnetic, mechanical or other types. Such pumps can be integral to or external to the system.

As discussed above, the analyte solution may also be circulated through one or more bifurcation channels 306 (i.e., a distribution manifold). These channels may couple a mixing channel and analyte chamber through two or more conduits that divide the flow path of the analyte solution. For example, a first bifurcation channel may include a first pair of ports that divide analyte solution leaving a mixing channel into two flow paths. Each the distal ends of each port may be coupled to a third and fourth pair of ports that further subdivide the analyte solution into four separate flow paths. Bifurcation channels that divide the circulation of analyte solution into two, three, four, five, six, seven, eight, etc., paths may be used.

The method 300 may further include binding analyte in the analyte solution to binding site 308 on a surface of the analyte chamber exposed to the solution. As noted above, examples of binding sites include sites formed with a strand of a nucleic acid that selectively hybridizes with a complementary nucleic acid strand in the analyte solution.

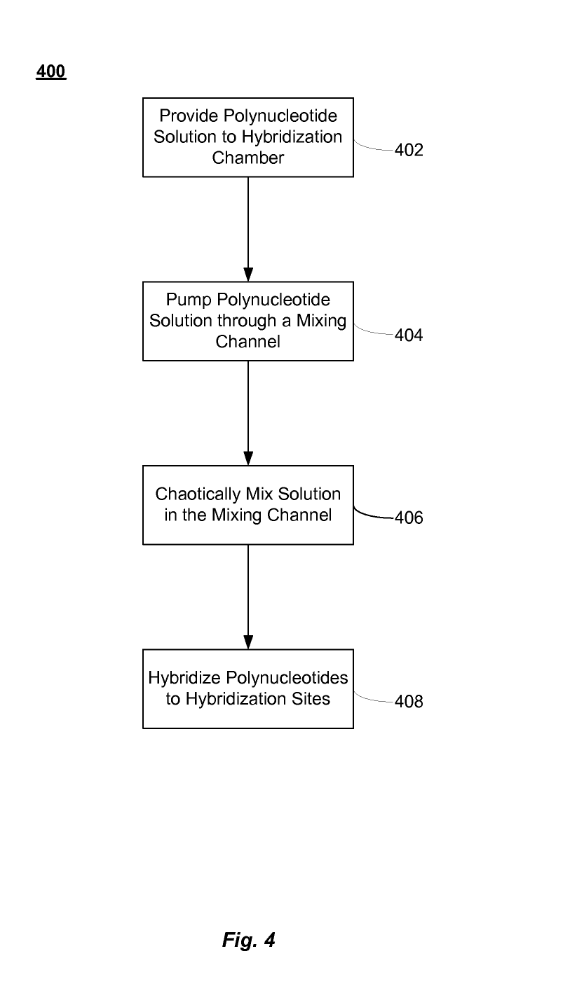

Details of a method 400 that is specifically directed to hybridizing polynucleotides in a solution of polynucleotides described with reference to FIG. 4. The method 400 includes providing the polynucleotide solution to a hybridization chamber 402. The initial solution may then be pumped though a mixing channel 404 to chaotically mix the solution in the mixing channel 406. Like other analytes, the pumping and chaotic mixing of the polynucleotide solution increases its homogeneity and reduces the formation of non-homogeneously mixed pockets of polynucleotides in the hybridization chamber(s).

The chaotically mixed polynucleotides are exposed to hybridization sites at a faster rate than if only static mixing were occurring in the hybridization chamber. This results in the polynucleotides hybridizing to the hybridization sites 408 faster, and increasing the signal from hybridized sites after a given period of mixing and hybridization. Additional details of nucleotide hydridizations and their applications are found in U.S. Pat. Pub. No. 2005/0196785, filed Jan. 5, 2005, and titled “COMBINATIONAL ARRAY FOR NUCLEIC ACID ANALYSIS”, by Quake et al, and U.S. Pat. Pub. No. 2005/0147992, filed Oct. 18, 2004, and titled “METHODS AND APPARATUS FOR ANALYZING POLYNUCLEOTIDE SEQUENCES”, by Quake et al, of which the entire contents of both published applications is herein incorporated by reference for all purposes.

It will be appreciated that a binding or annealing step is sometimes following by art-known washing steps to reduce background from non-specific binding. Specific binding can be detected using art-known methods. Detection methods include any detection method suitable for the particular analyte and ligand. Illustrative detection methodologies suitable for use with the present microfluidic devices include, but are not limited to, light scattering, multichannel fluorescence detection, infra-red, UV and visible wavelength absorption, luminescence, differential reflectivity, and confocal laser scanning. Additional detection methods that can be used in certain applications include, without limitation, scintillation proximity assay techniques, radiochemical detection, fluorescence polarization, fluorescence correlation spectroscopy (FCS), time-resolved energy transfer (TRET), fluorescence resonance energy transfer (FRET) and variations such as bioluminescence resonance energy transfer (BRET), electrical resistance, resistivity, impedance, and voltage sensing. In one embodiment the array is removed from the system prior to the detection step. Alternatively detection can be carried out using the array in situ.

### EXPERIMENTAL

Comparison of Dynamic and Static Hybridizations

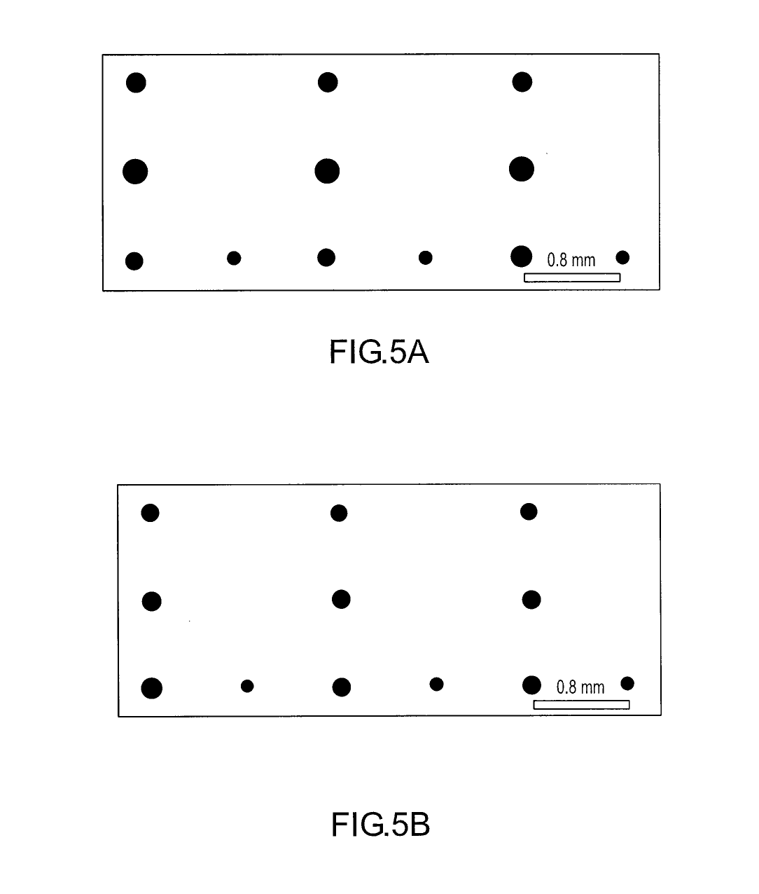

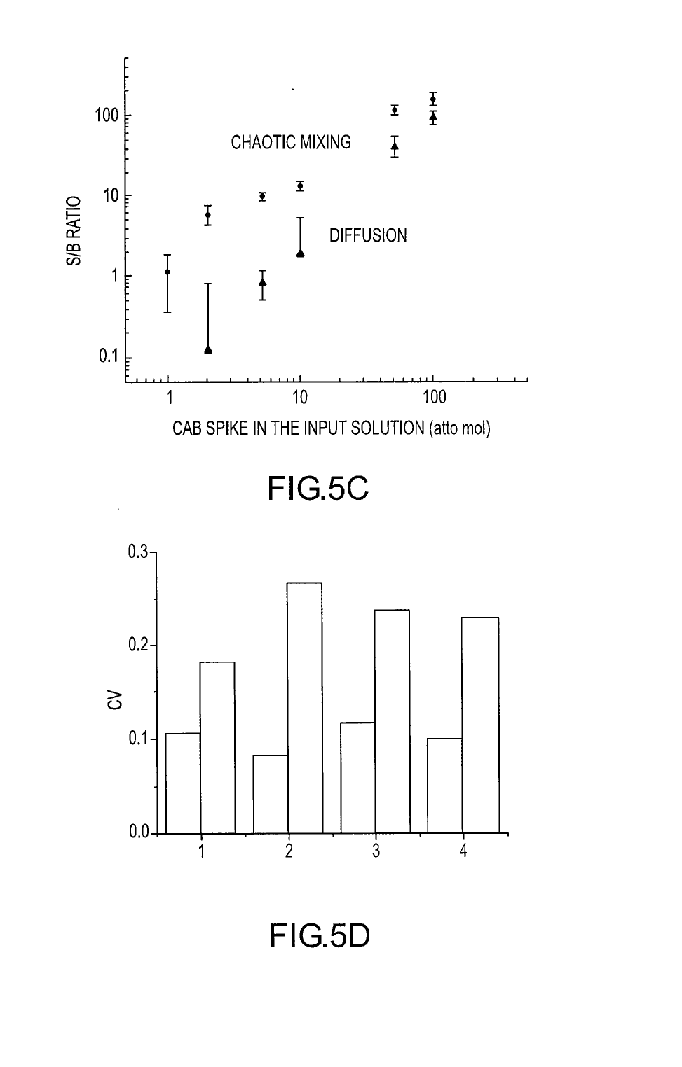

A series of dynamic and static (control) hybridizations were conducted for the purpose of comparison. Two PDMS devices were sealed onto a single home-spotted microarray slide, covering two areas of the probes with identical pattern. As shown in FIGS. 5A & B, each area consisted of four identical blocks. Each block includes 18 features (6 spotting solutions×repetition 3). Six of them are invisible negative control features. We prepared Cy3-labeled cDNA from the C2C12 mouse skeletal muscle cell line, adding A. thaliana Cab spike (cat # 2552201, Stratagene) as a positive control. Details of total RNA isolation, mRNA extraction, and the reverse transcription protocols are as described in Williams B A, Gwirtz R M, Wold B J. 2004. Nucleic Acids Research 32: e8 1, the entire contents of which is herein incorporated by reference for all purposes.

The Cy3-labeld cDNA sample was diluted into a series of solutions and then aliquoted. The solutions were spin-dried under vacuum and kept at 4° C. before use. Two identical aliquots were used to prepare the hybridization solutions with the ArrayHyb buffer purchased from Sigma-Aldrich Co. They were respectively loaded into either one of the PDMS devices sealed on the same slide, which had been mounted on the flat bed of the thermocycler (PTC-200, MJ Research). Dynamic hybridization was performed by actuating all the peristaltic pumps in one of the devices; while static hybridization was performed in the other device as a control experiment. After a hybridization of 2 hours at 52° C., the PDMS devices were peeled away from the slide. It was immediately removed into a plastic tube for programmed post-hybridization washing (AdvaWash 400, Advalytix). Then the slide was spin-dried using a centrifuge (5804R, Eppendorf) and scanned (ArrayWorx, Applied Precision LLC) to obtain fluorescent images.

Hybridization using microfluidic chaotic mixing produced much stronger signals (FIG. 5A), compared with the static control (FIG. 5B). And the new approach achieved better sensitivity than the conventional method, as shown in FIG. 5C. When the input molecules of the CAB spike was reduced to 10 attomol, the signal to noise ratio of static method was slightly larger than 1, indicating that the signals were almost indistinguishable from the background at that point. However, the S/N ratio of dynamic hybridization did not collapse to 1 until the input CAB molecules further decreased to 1 attomol, an enhancement of one order of magnitude in terms of sensitivity. To our knowledge, that sensitivity level was better than any other reported method designed for active mixing in hybridization.

In addition, the new approach of active mixing brought down the spot-to-spot fluctuation of signals. Table 1 shows the spot-to-spot coefficients of variation (CV) (n=12) of dynamic versus static hybridization:

TABLE 1
The spot-to-spot Coefficients of Variation of Dynamic Vs.
Static Mixing

| Oligonucleotide Probe | Dynamic Mixing | Static Mixing |
|---|---|---|
| Myogenin Sense | 0.11 | 0.18 |
| Muscle Creatine Kinase | 0.08 | 0.27 |
| (MCK) sense | | |
| myosin light-chain sense | 0.12 | 0.24 |
| chlorohlyll a/b-binding | 0.10 | 0.23 |
| protein (CAB) positive | | |
| control | | |

As Table 1 shows, the coefficients of variation (CV) of the dynamic hybridization were nearly reduced to a half of the values of the conventional static method, as also shown by FIGS. 5C & D. The results of the hybridization kinetics (FIG. 5D) also showed that dynamic mixing consistently produced signals with higher S/N ratios than the static method. The signal dynamic hybridization for 2 h was nearly twice that from the static control hybridized for 6 h. We notice that the signals from both methods decreased after >6 h hybridization, which might be attributed to partial dehydration of the arrays, as PDMS is permeable to water vapor.

The slide-to-slide variation was checked by independent hybridization experiments. The CV values (n=2) of dynamic hybridization were less than 13%, while those of static method were less than 27%. Therefore the above enhancement of signals was repeatable.

The evaluate the separate contributions of the circulatory motion of the fluid and chaotic to the signal enhancement, we performed side-by-side comparison experiments that included an additional hybridization control with clued circulation but without chaotic mixing. Identical aliquots of DNA target solutions were hybridized at 52° C. for 2 h under three distinct conditions: Static (control 1), simple fluid circulation using the devices without the herring-bone protrusions (control 2), and fluid circulation with chaotic mixing. The experimental results listed below in Table 2 clearly show that chaotic mixing played a significant part in the overall signal enhancement. Simple circulation of the fluid increased the signal intensity to 1.6-2.3 fold those obtained from the static control, whereas circulation with chaotic mixing improved the signals 3.4-6.9 fold. Therefore, microfluidic chaotic mixing has a major effect on the mass transfer of DNA targets to a solid reactive boundary by effectively homogenizing the solution.

TABLE 2
Increase (x-times) in the background-subtracted fluorescence of
dynamic compared with static hybridization

| | Without | With Chaotic |
|---|---|---|
| Oligonucleotide Probe | Chaotic Mixing | Mixing |
| Myogenin Sense | 2.1 | 4.2 |
| Muscle Creatine Kinase | 1.6 | 6.9 |
| (MCK) sense | | |
| myosin light-chain sense | 2.2 | 3.4 |
| chlorohlyll a/b-binding | 2.3 | 4.5 |
| protein (CAB) positive | | |
| control | | |

Microarray Compatibility Tests

The compatibility of microfluidic chaotic mixing devices with commercial microarray was demonstrated. The tests showed that microfluidic chaotic mixing can improve hybridization of high density microarray experiments. We fabricated a PDMS device with similar geometry, except that the hybridization chambers of the new device were larger (7.5 mm×36 mm×65 microns, 35 μL). It was sealed onto a commercially available microarray (18 K printed oligonucleotides 70-mer for mouse genome, J. David Gladstone Institutes in UCSF) and baked in an oven at 80° C. for three hours before use. Approximately 9,500 spots on the slide were accessible within the hybridization chambers. All the experimental procedures followed the previous description except that two identical microarray slides were put into use for comparison.

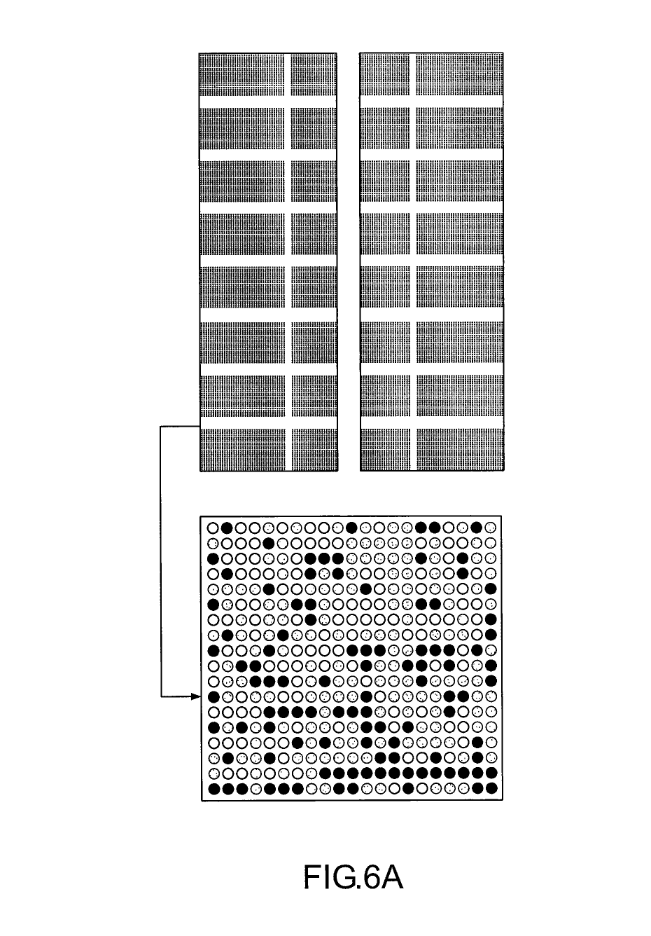

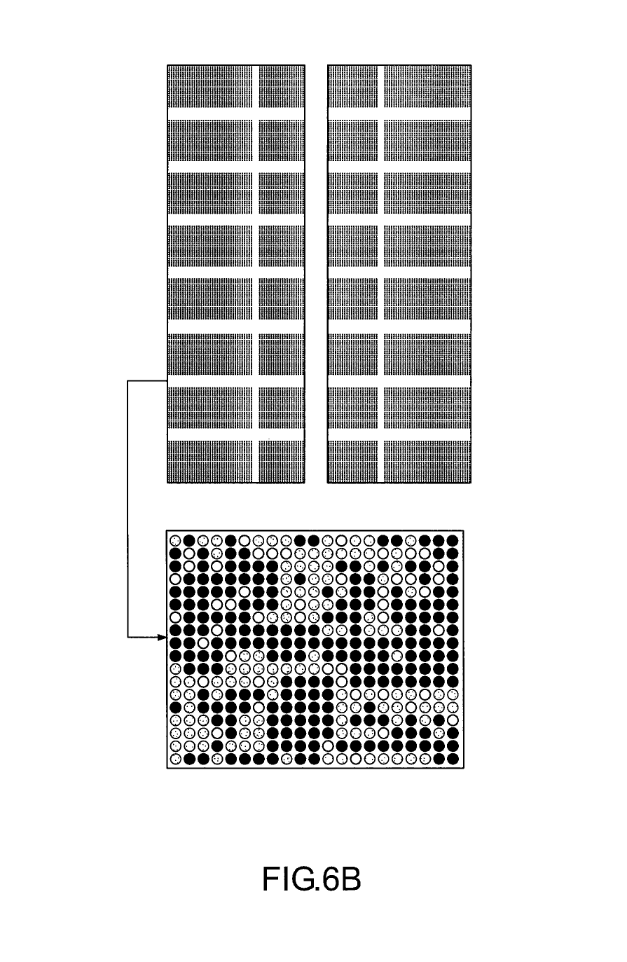

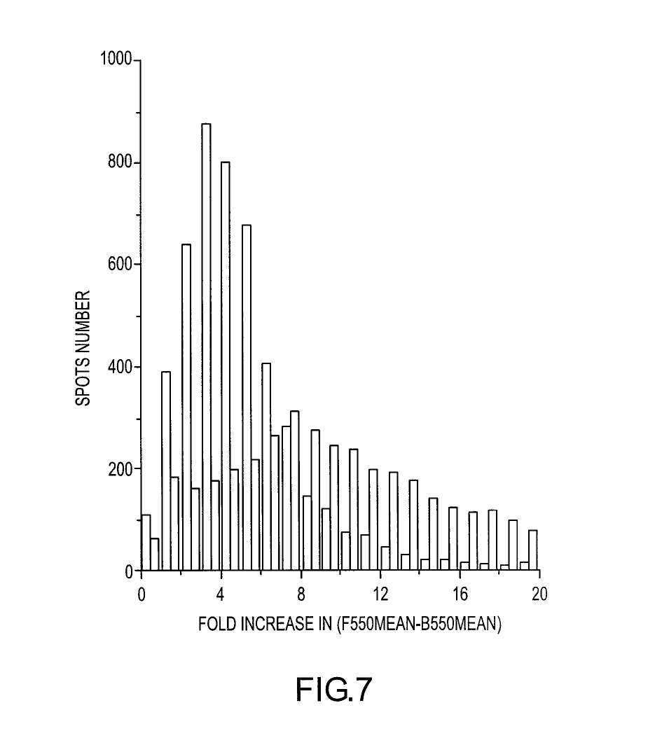

The signals from hybridization using microfluidic chaotic mixing were dramatically enhanced, as showed by the FIG. 6A and FIG. 6B (control). For individual spots, we calculated the fold increase in the background-subtracted fluorescence by dynamic mixing over static method, and then made a histogram (FIG. 7). The peak of the red columns (input sample concentration 1.6 ng/ul) was located between 3 to 4 fold. When the input Cy3 labeled cDNA was diluted to 0.8 ng/ul (represented by the blue columns), the peak shifted to 7 to 8 fold. That was consistent with our previous observation: The fold increase of signals by dynamic over static hybridization became larger in lower concentration of input samples.

In literature, there are different numbers of fold increase reported with various control experiments, mostly ranging from 2 to 5 fold. We further calculated the coefficients of correlation of background-subtracted signals in the above two concentrations. The value of R was 0.78 for dynamic hybridization, while it was 0.24 for static method. So microfluidic chaotic mixing helped produce more predictable results than the conventional method.

Percentage of cDNA Molecules Hybridized Using Chaotic Mixing

It was intriguing to examine how many cDNA molecules were actually hybridized onto the slide from the solution. We manually deposited volume-defined droplets of Cy3 labeled cDNA molecules onto the slide using microcaps (0.2 ul, Drummond). Then we obtained the standard curve of the relation between the fluorescence intensity of pixels and the total amount of deposited molecules. We calculated the percentage of hybridized Cab molecules out of the input amount of the spike, based on the data of home-spotted microarray experiments. The analysis was based on 2-h hybridization experiments. The data shown below in Table 3 reveal that only a small percentage of target DNA molecules were actually hybridized with the static method, with a large portion of them remaining in solution. Dynamic mixing can increase the percentage of molar-hybridization events several fold.

TABLE 3
Percentages of Hybridized CAB Molecules out of the Total Number
of Initial Spikes

| | Dynamic Hybridization | Static Hybridization |
|---|---|---|
| Input CAB | [%] | [%] |
| 55 | 9.4 | 1.3 |
| 28 | 4.1 | 0.66 |
| 5.5 | 1.2 | 0.20 |
| 2.8 | 1.6 | 0.20 |
| 1.1 | 1.9 | 0.54 |

The yield of the labeling reaction was about 54% according to absorption measurements. The full length of the CAB spikes (500 base pairs) was used in the above estimation. This represents a conservative lower limit as the hexamer-priming reaction may yield a distribution of cDNA lengths.

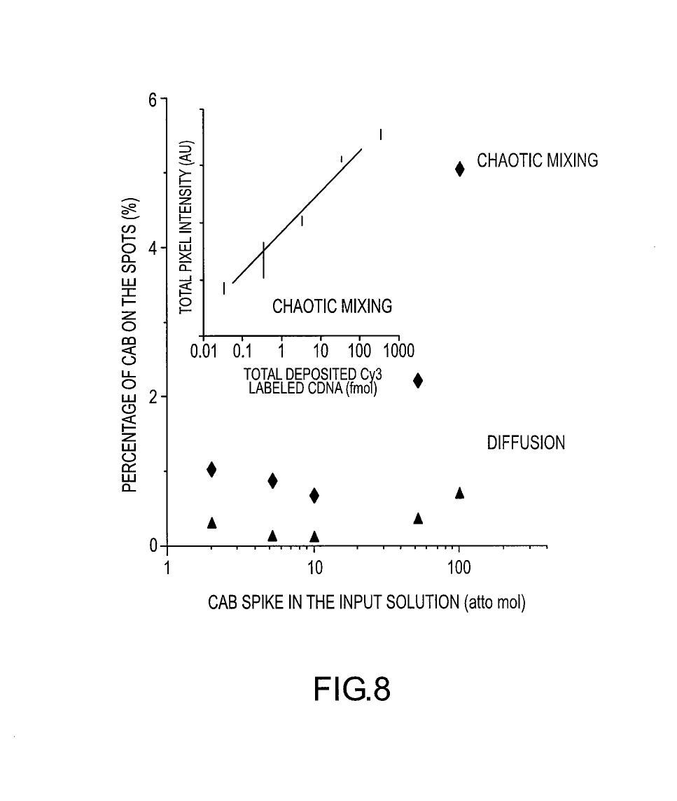

On average microfluidic chaotic mixing increased the hybridization events 6 to 8 fold, compared with the conventional static method (FIG. 8). We observed larger fold increase, but relatively smaller percentages (dynamic or static) than the reported values because those experiments were hybridized for longer time (>4 hr), and their input sample (at least 60 times more concentrated) was possibly not in the comparable concentration range. The data revealed that only a small percentage of target DNA molecules were actually hybridized with the conventional method, while a large number of them remained in the solution. Dynamic mixing increased the percentage of molar hybridization events by several fold.

A description of the above-described experiments, and a discussion of the results, can also be found in a published communication titled “Enhanced Signals and Fast Nucleic Acid Hybridization by Microfluidic Chaotic Mixing” by J. Liu et al, Angew. Chem. Int. Ed., 2006, 45, 3618-3623, the entire contents of which are herein incorporated by reference for all purposes.

Having described several embodiments, it will be recognized by those of skill in the art that various modifications, alternative constructions, and equivalents may be used without departing from the spirit of the invention. Additionally, a number of well known processes and elements have not been described in order to avoid unnecessarily obscuring the present invention. Accordingly, the above description should not be taken as limiting the scope of the invention.

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the channel” includes reference to one or more channels and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

What is claimed is:

1. A microfluidic nucleic acid hybridization system comprising:
a first reaction chamber to hold an analyte solution comprising nucleic acids;
a first mixing channel in fluid communication with the chamber, wherein the channel includes a textured surface to mix the analyte solution;
a pump coupled to the mixing channel to circulate the analyte solution through the reaction chamber and the mixing channel;
an input port in fluid communication with the mixing channel and the reaction chamber to supply the analyte solution to the microfluidic system, wherein the input port can be closed to create a closed circulation path for the analyte solution through the reaction chamber and the mixing channel.

2. The microfluidic nucleic acid hybridization system of claim 1, wherein the system comprises analyte binding sites present in the chamber.

3. The microfluidic nucleic acid hybridization system of claim 2, wherein the analyte binding sites comprise a nucleic acid microarray.

4. The microfluidic nucleic acid hybridization system of claim 2, wherein the analyte binding sites are formed on an interior surface of the chamber.

5. The microfluidic nucleic acid hybridization system of claim 2, wherein the analyte binding sites are formed on a removable substrate positioned inside the chamber.

6. The microfluidic nucleic acid hybridization system of claim 1, wherein the textured surface of the mixing channel comprises a plurality of groves or protrusions with orientations that form angles relative to a principle direction of fluid flow.

7. The microfluidic nucleic acid hybridization system of claim 1, wherein the textured surface comprises chevron-shaped groves or protrusions.

8. The microfluidic nucleic acid hybridization system of claim 1, wherein the turbulence generating pattern comprises a herring-bone pattern.

9. The microfluidic nucleic acid hybridization system of claim 1, wherein the pump is a peristaltic pump.

10. The microfluidic nucleic acid hybridization system of claim 1, wherein the system comprises a distribution manifold coupled between the chamber and the mixing channel, wherein the manifold comprises a plurality of branches to divide the analyte solution flowing through the manifold.

11. The microfluidic nucleic acid hybridization system of claim 10, wherein the distribution manifold comprises a bifurcation channel with a first and second branch that divides the analyte solution flowing through the channel.

12. The microfluidic nucleic acid hybridization system of claim 10, wherein the distribution manifold comprises a first subdividing bifurcation channel coupled to the first branch, which further divides the analyte solution between at least two more branches, and a second subdividing bifurcation channel coupled to the second branch, which further divides the analyte solution between at least two more branches.

13. The microfluidic nucleic acid hybridization system of claim 1, wherein the input port is in fluid communication with a closable rubber gasket or elastomeric valve that reversibly closes the port.

14. The microfluidic nucleic acid hybridization system of claim 1, wherein the system comprises a output port in fluid communication with the reaction chamber to remove analyte solution from the system.

15. The microfluidic nucleic acid hybridization system of claim 1, wherein the system comprises:
two or more reaction chambers containing analyte binding sites, wherein each chamber is connected to another chamber via a mixing channel so that fluid can circulate in a closed loop through said chambers and channels.

16. The microfluidic nucleic acid hybridization system of claim 15, wherein the system comprises one or more additional ports to supply or remove the analyte solution from the microfluidic system, wherein said ports can be closed to isolate the system and form a closed loop through which fluid can circulate.

17. The microfluidic nucleic acid hybridization system of claim 15, wherein the system comprises a plurality of pumps coupled to the mixing channels to circulate the analyte solution through the system.

18. The microfluidic nucleic acid hybridization system of claim 1, wherein the system further comprises:
a second reaction chamber in fluid communication with the first mixing channel; and
a second mixing channel in fluid communication with the first and second reaction chambers,
wherein the first and second channels and reaction chambers form a fluid flow path to mix and circulate the analyte solution between the first and second reaction chambers.

19. The microfluidic nucleic acid hybridization system of claim 18, wherein the second mixing channel includes a textured surface to facilitate the chaotic mixing of the analyte solution.

20. The microfluidic nucleic acid hybridization system of claim 19, wherein the textured surface in the second channel comprises a plurality of groves or protrusions with orientations that form angles relative to a principle fluid flow direction.

21. The microfluidic nucleic acid hybridization system of claim 19, wherein the textured surface in the second mixing channel comprises a herring-bone pattern.

22. The microfluidic nucleic acid hybridization system of claim 1, wherein the first mixing channel comprises an elastomeric material.

23. The microfluidic nucleic acid hybridization system of claim 1, wherein the first reaction chamber comprises an elastomeric material.

24. A method of chaotically mixing and hybridizing a nucleic acid solution in a microfluidic system, the method comprising:
providing the nucleic acid solution to a first reaction chamber, wherein the reaction chamber contains nucleic acid hybridization sites;
circulating the nucleic acid solution a plurality of times through a mixing channel coupled to the reaction chamber, wherein the solution is mixed as it flows across a textured surface of the mixing channel to mix the solution; and
hybridizing nucleic acids in the solution to one of the hybridization sites.

25. The method of claim 24, wherein the method comprises circulating the nucleic acid solution from the first reaction chamber to a second reaction chamber through the mixing channel.

26. The method of claim 24, wherein the method comprises binding the nucleic acids to hybridization sites in both the first and second analyte chambers.

27. The method of claim 24, wherein the textured surface comprises a plurality of groves or protrusions with orientations that form angles relative to a principle fluid flow direction.

28. The method of claim 27, wherein the textured surface comprises a herring-bone pattern.

29. The method of claim 24, wherein the nucleic acid solution is circulated with a peristaltic pump coupled to the mixing channel.

30. A microfluidic nucleic acid hybridization system comprising:
a glass substrate having an array of nucleic acids on a top surface of the substrate;
a first elastomeric layer attached to the top surface of the glass substrate, wherein the elastomeric layer has a first and second reaction chamber formed therein, and wherein the top surface of the substrate forms an inside surface of each reaction chamber;
a mixing channel that is also formed in the first elastomeric layer, where the mixing channel is in fluid communication with the first and second reaction chamber, and wherein the mixing channel has a textured surface with a herring-bone pattern to mix a nucleic acid solution flowing through the channel;
a second elastomeric layer formed on the first elastomeric layer, wherein the second elastomeric layer has a series of control channels that activate a microfluidic peristaltic pump in the mixing channel; and
an input port in fluid communication with the mixing channel and the reaction chamber to supply the nucleic acid solution to the microfluidic system, wherein the input port is coupled to a closable microvalve that creates a closed circulation path for the nucleic acid solution between the reaction chamber and the mixing channel.
